(19) United States
(12) Patent Application Publication
(10) Pub. No.: US 2022/0265131 A1
(43) Pub. Date: Aug. 25, 2022

(54) ENDOSCOPE DISTAL END STRUCTURE AND ENDOSCOPE

(71) Applicant: OLYMPUS CORPORATION, Tokyo (JP)

(72) Inventor: Nau SATAKE, Yokohama-shi (JP)

(73) Assignee: OLYMPUS CORPORATION, Tokyo (JP)

(21) Appl. No.: 17/731,717

(22) Filed: Apr. 28, 2022

Related U.S. Application Data
(63) Continuation of application No. PCT/JP2020/002836, filed on Jan. 27, 2020.

(51) Int. Cl. A61B 1/05; A61B 1/00

(52) U.S. Cl. CPC A61B 1/05; A61B 1/00114; A61B 1/00124

(57) ABSTRACT

An endoscope distal end structure includes: an image sensor; a composite cable that is formed by bundling a plurality of signal cables; a substrate that includes a plurality of electrodes for connecting respective core wires of the plurality of signal cables; a cable fixing portion configured to hold the plurality of signal cables that are exposed from an end portion of the composite cable and the end portion of the composite cable; a sealing resin configured to protect connection portions between the core wires of the plurality of signal cables and the plurality of electrodes; and a shrinkable tube. The cable fixing portion includes a first fixing portion that is arranged on a surface of the substrate, and the first fixing portion includes grooves that are divided by wail portions, the grooves being configured to house the respective signal cables.

### CROSS-REFERENCE TO RELATED APPLICATION

This application is a continuation of International Application No. PCT/JP2020/002836, filed on Jan 27, 2020, the entire contents of which. are incorporated herein by reference.

### BACKGROUND

### 1. Technical Field

The present disclosure relates to an endoscope distal end structure and an endoscope.

### 2. Related Art

In the related art, as endoscope is configured such that an insertion portion is inserted into a subject, such as a patient, and an imaging module acquires image data of an inside of the subject. The image data acquired by the imaging module is transmitted, via a signal cable, to an information processing apparatus that is electrically connected to the imaging module, and a periphery of a connection portion of the signal cable is filled with a sealing resin for protecting the connection portion (for example, see Japanese Laid-open Patent Publication No. 2008-118568).

### SUMMARY

In some embodiments, an endoscope distal end structure includes: an image sensor configured to capture an image of an inside of a subject and generate an image signal; a composite cable that is formed by bundling a plurality of signal cables by an electrical insulating outer casing; a substrate that includes a plurality of electrodes for connecting respective core wires of the plurality of signal cables, the substrate being configured to electrically connect the image sensor and the plurality of signal cables; a cable fixing portion that is formed of an elastic body, the cable fixing portion being configured to hold the plurality of signal cables that are exposed from an end portion of the composite cable and the end. portion of the composite cable; a sealing resin configured to protect connection portions between the core wires of the plurality of signal cables and the plurality of electrodes; and a shrinkable tube configured to cover peripheries of the image sensor and the, cable fixing portion. The cable fixing portion includes a first fixing portion that is arranged on a surface of the substrate, the electrodes are formed on the surface of the substrate, the first fixing portion being configured to cover at least a part of peripheries of the plurality of signal cables, and the first fixing portion includes grooves that are divided by wall portions, the grooves being configured to house the respective signal cables.

In some embodiments, an endoscope includes: an insertion portion configured to be inserted into a subject; an image sensor that is arranged on the insertion portion, the image sensor being configured to capture an image of an inside of the subject and generate an image signal; a composite cable that is formed by bundling a plurality of signal cables by an electrical insulating outer casing; a substrate that includes a plurality of electrodes for connecting respective core wires of the plurality of signal cables, the substrate being configured to electrically connect the image sensor and the plurality of signal cables; a cable fixing portion that is formed of an elastic body, the cable fixing portion being configured to hold the plurality of signal cables that are exposed from an end portion of the composite cable and the end portion of the composite cable; a sealing resin configured to protect connection portions between the core wires of the plurality of signal cables and the plurality of electrodes; and a shrinkable tube configured to cover peripheries of the image sensor and the cable fixing portion. The cable fixing portion includes a first fixing portion that is arranged on a surface of the substrate, the electrodes being formed on. the surface of the substrate, the first fixing portion being configured to cover at least a part of peripheries of the plurality of signal cables, and the first fixing portion includes grooves that are divided by wall portions, the grooves being configured to house the respective signal cables.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an entire configuration of an endoscope system according to a first embodiment of the disclosure;

FIG. 2A is a cross-sectional view of an endoscope distal end structure according to the first embodiment of the disclosure taken in an optical axis direction;

FIG. 2B is a cross-sectional view taken along a line A-A in FIG. 2A;

FIG. 2C is a cross-sectional view taken along a line B-B in FIG. 1A;

FIG. 3A is a side view of a cable fixing portion that is used. in the endoscope distal end structure according to the first embodiment of the disclosure;

FIG. 3B is a top view of the cable fixing portion. in FIG. 3A;

FIG. 3C is a front view of the cable fixing portion in FIG. 3A;

FIG. 3D is a back view of the cable fixing portion in FIG. 3A;

FIG. 4 is a cross-sectional view of an endoscope distal end structure according to a first modification of the first embodiment of the disclosure;

FIG. 5 is a cross-sectional view of an endoscope distal end structure according to a second modification of the first embodiment of the disclosure;

FIG. 6 is a cross-sectional view of an endoscope distal end structure according to a third modification of the first embodiment of the disclosure;

FIG. 7 is a cross-sectional view of a groove is a cable fixing portion of an endoscope distal end structure according to a fourth modification of the first embodiment of the disclosure;

FIG. 8 is a cross-sectional view of a groove in a cable fixing portion of an endoscope distal end structure according to a fifth modification of the first embodiment of the disclosure;

FIG. 9A is a side view of a cable fixing portion and an upper cable fixing portion that are used in an endoscope distal end structure according to a second embodiment of the disclosure;

FIG. 9B is a side view of the upper cable fixing portion;

FIG. 10A is a cross-sectional view of the endoscope distal end structure according to the second embodiment of the disclosure;

FIG. 10B is a cross-sectional view of the endoscope distal end structure according to the second embodiment of the disclosure;

FIG. 11 is a side view of a cable fixing portion and an upper cable fixing portion that are used in an endoscope distal end structure according to a modification of the second embodiment of the disclosure; and

FIG. 12 is a top view of as endoscope distal end structure according to a third embodiment of the disclosure.

### DETAILED DESCRIPTION

As modes for carrying out the disclosure (hereinafter, referred to as “embodiments”), an endoscope system including an endoscope distal end structure will be described below. The disclosure is not limited by the embodiments below. Furthermore, in the drawings referred to in the following description, shapes, sizes, and positional relationships are only schematically illustrated so that the contents of the disclosure may be understood. Namely, the disclosure is not limited to only the shapes, the sizes, and. the positional relationships illustrated in the drawings. Moreover, the drawings may include portions that have different dimensions or dimensional ratios.

### First Embodiment

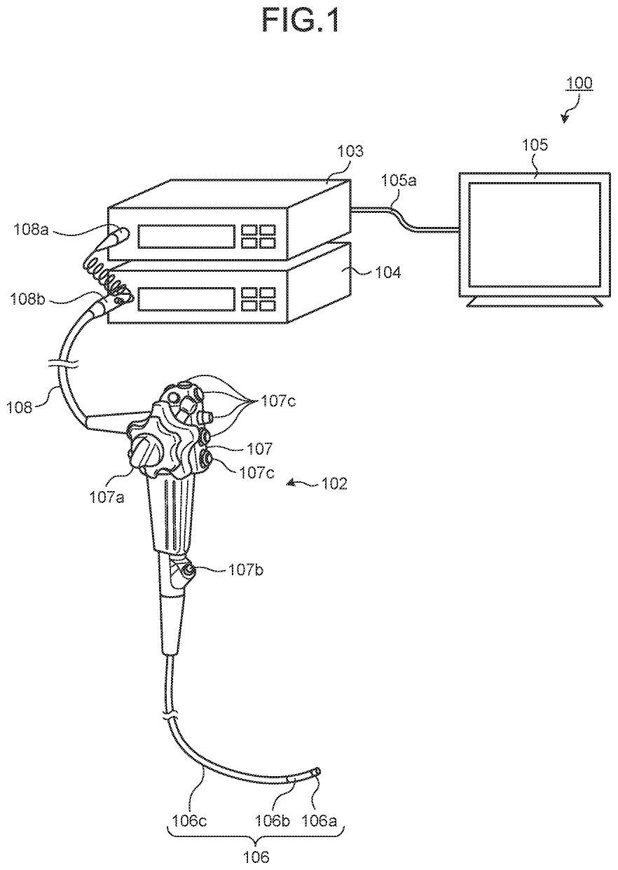

FIG. 1 is a diagram schematically illustrating an entire configuration of an endoscope system 100 according to a first embodiment of the disclosure. As illustrated in FIG. 1, the endoscope system 100 according to the present embodiment includes an endoscope 102 that is introduced into a subject, that captures an image of an inside of a body of the subject, and that generates an image signal of the inside of the subject, an information processing apparatus 103 that performs predetermined image processing on the image signal captured by the endoscope 102 and that controls each of units of the endoscope system 100, a light source device 104 that generates illumination light of the endoscope 102, and a display device 105 that displays an image of the image signal that is subjected to the image processing by the information processing apparatus 103.

The endoscope 102 includes an insertion portion 106 that is inserted into the subject, an operating unit 107 that is arranged on a proximal end side of the insertion portion 106 and that is gripped by the operator, and a flexible universal cord 108 that extends from the operating unit 107.

The insertion portion 106 is realized by using a light guide formed of an illumination fiber, an electrical cable, an optical fiber, or the like. The insertion portion 106 includes a distal end portion 106a that includes an endoscope distal end structure to be described later, a bending portion 106b that is freely bendable, and a flexible tube portion 106c that is arranged on a proximal end side of the bending portion 106b and that has flexibility.

The operating unit 107 includes a bending knob 107a that causes the bending portion 106b to bend in a vertical direction and in a horizontal direction, a treatment tool insertion portion 107b that allows a treatment tool, such as a biopsy forceps or a laser scalpel, to be inserted into a body cavity of the subject, and a plurality of switch portions 107c for performing operation of peripheral devices, such as the information processing apparatus 103, the light source device 104, an air supply device, a water supply device, a gas supply device, and a bending tube. The treatment tool that is inserted through the treatment tool insertion portion 107b gets out of an opening portion that is arranged on a distal end of the insertion portion 106 through an internally-arranged treatment tool channel.

The universal cord 108 is configured with a light guide formed of an illumination fiber, a cable, or the like. The universal cord 108 is branched at a proximal end thereof, where one branched end portion serves as a connector 108a and another proximal end serves as a connector 108b. The connector 108a is detachably attachable to a connector of the information processing apparatus 103. The connector 108b is detachably attachable to the light source device 104. The universal cord 108 allows the illumination light that is emitted from the light source device 104 to propagate to the distal end portion 106a via the connector 108b and the light guide formed of the illumination fiber. Further, the universal cord 108 transmits an image signal that is captured by an imaging apparatus (to be described later) to the information processing apparatus 103 via the cable and the connector 108a.

The information processing apparatus 103 performs predetermined image processing on an image signal output from the connector 108a, and controls the entire endoscope system 100.

The light source device 104 is configured with a light source that emits light, a condenser lens, or the like.

The light source device 104 emits light from the light source and supplies the light as illumination light, which is to be applied to the inside of the subject that is an imaging object, to the endoscope 102 that is connected via the connector 108b and the light guide formed of the illumination fiber of the universal cord 108, under the control of the information processing apparatus 103.

The display device 105 is configured with a display made of liquid crystal or organic electro luminescence (EL), for example. The display device 105 displays various kinds of information including an image that is subjected to the predetermined image processing by the information processing apparatus 103, via a video cable 105a. With this configuration, an operator is able to observe and determine a symptom of a desired position inside the subject by operating the endoscope 102 while watching an image (in-vivo image) that is displayed by the display device 105.

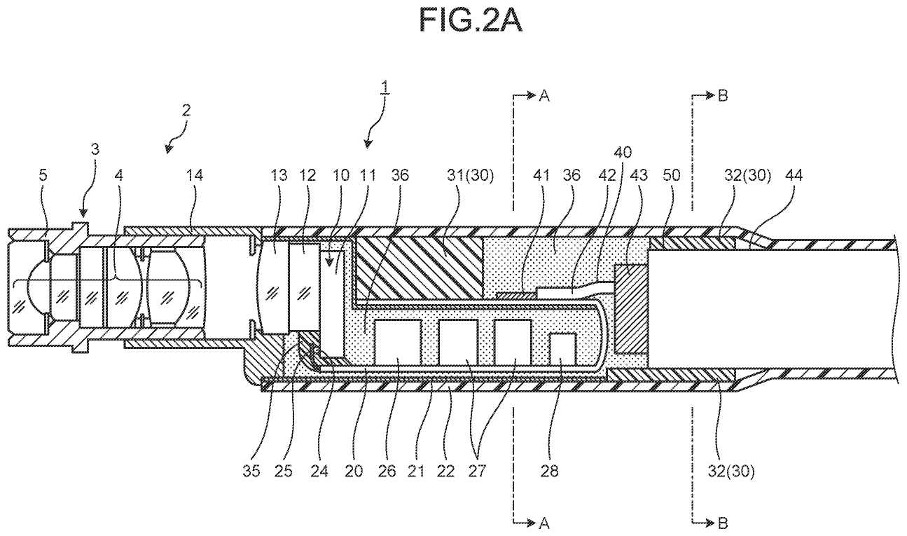

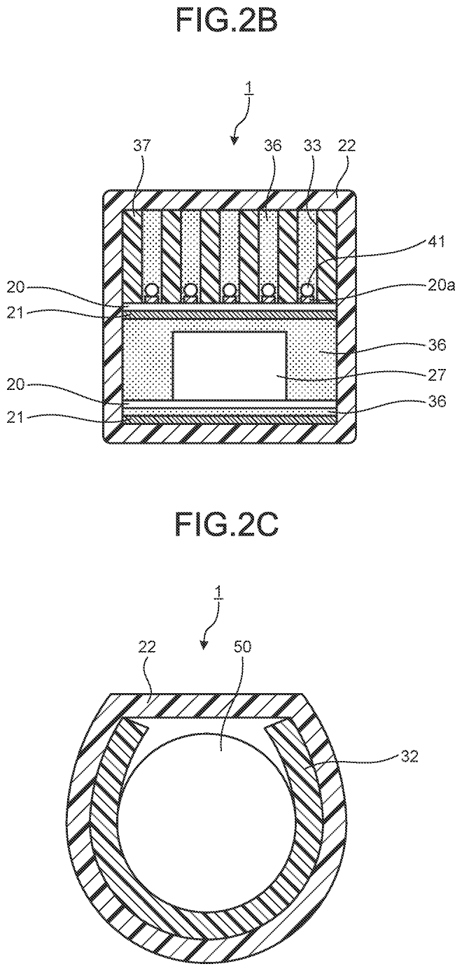

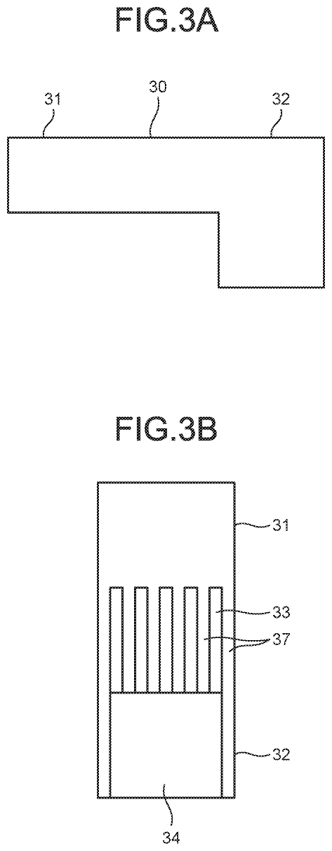

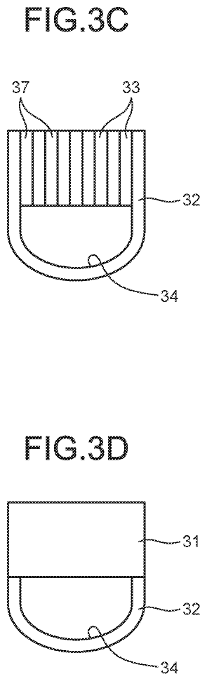

The endoscope distal end structure that is arranged in the distal end portion 106a of the endoscope system 100 will be described in detail below. FIG. 2A is a cross-sectional view of an endoscope distal end structure 1 according to the first embodiment of the disclosure taken along an optical axis direction. FIG. 2B is a cross-sectional view taken along a line A-A in FIG. 2A. FIG. 2C is a cross-sectional view taken along a line B-B in FIG. 2A. FIG. 3A is a side view of a cable fixing portion 30 that is used in the endoscope distal end structure 1 according to the first embodiment of the disclosure. FIG. 3B is a top view of the cable fixing portion 30 in. FIG. 3A. FIG. 3C is a front view of the cable fixing portion 30 in FIG. 3A. FIG. 3D is a back view of the cable fixing portion 30 in FIG. 3A.

The endoscope distal end structure 1 includes an imaging module 2 that captures an image of the inside of the subject and generates an image signal, a composite cable 50 that is formed by bundling a plurality of signal cables 40 by an electrical insulating outer casing 44, the cable fixing portion 30 that is formed of an elastic body and holds the plurality of signal cables 40 that are exposed from an end portion of the composite cable 50 and the end portion of the composite cable 50, a sealing resin 36 that protects connection portions of the plurality of signal cables 40, and a heat shrinkable tube 22 that covers and protects peripheries of a proximal end side of the imaging module 2 and the cable fixing portion 30. Meanwhile, in the present specification, a direction that is the same as a longitudinal direction of the endoscope 102 and in which the insertion portion 106 is inserted will be referred to as an “insertion direction”, a side on which the insertion portion 106 is inserted when viewed from the operating unit. 107 will be referred to as a “distal end side”, and a side opposite to the distal end side will be referred to as a “proximal end side”.

The imaging module 2 includes an objective optical system unit 3 and an imaging unit 10 that is arranged on a rear side of the objective optical system unit 3.

The objective optical system unit 3 includes an objective lens group 4 formed of a plurality of lenses, and an objective lens frame 5 that holds the objective lens group 4. The objective lens frame 5 is held on the distal end portion 106a by being fitted to a distal end frame of the endoscope 102.

A solid state image sensor frame 14 is fitted to a proximal end side of the objective lens frame 5, and the imaging unit 10 is held on the objective optical system unit 3 via the solid state image sensor frame 14. The objective optical system unit 3 forms an image of an incident light that has entered via the objective lens group 4 onto a light receiving surface of a solid state image sensor 11 of the imaging unit 10.

The imaging unit 10 includes the solid state image sensor 11, a cover glass 12 that protects the solid state image sensor 11, a centered glass 13, a substrate 20 on which electronic components, such as a transistor 26, a chip capacitor 27, and a chip resistance 28, are mounted, and a reinforcing frame 21 in which. an electronic component mounting portion of the substrate 20, the cover glass 12, and the solid state image sensor 11 are housed. The substrate 20 is a flexible substrate that is bendable.

A plurality of electrodes are arranged on a lower side of the light receiving surface of the solid state image sensor 11, and connected to an inner lead 25 of the substrate 20 via a bump 24. A periphery of a connection portion among the electrodes of the solid state image sensor 11, the inner lead 25, and the bump 24 is covered by a sealing resin 35.

The inner lead 25 is bent parallel to a bottom side of the solid state image sensor 11, and the substrate 20 is extended parallel to an optical axis of the objective optical system unit 3. A proximal end side of the substrate 20 relative to the electronic component mounting portion on which the transistor 26, the chip capacitor 27, and the chip resistance 28 are mounted is bent in an inverted C shape. A plurality of electrodes 20a (to be described later) to which the signal cables 40 are connected are arranged on a surface of the substrate 20 that is opposite to a surface on which the electronic components are mounted and that is located on the proximal end side relative to the electronic component mounting portion.

The reinforcing frame 21 is a square-shaped frame that is configured with a metallic thin plate and that has a step, and both end portions thereof in the longitudinal direction are opened. A distal end side of the reinforcing frame 21 has a size with which the solid state image sensor 11 can be housed, and a proximal end side has a size with which the electronic components can be housed. Further, an electrical insulating coating is applied to an entire inner peripheral surface of the reinforcing frame 21.

After inserting the substrate 20 into the opening of the reinforcing frame 21, fitting the reinforcing frame 21 to a periphery of the solid state image sensor 11 or the like, and filling the reinforcing frame 21 with the sealing resin 36 for adhesive fixing, the substrate 20 is bent along the shape of the reinforcing frame 21 and fixed to the reinforcing frame 21.

The composite cable 50 is formed of the plurality of signal cables 40 that transmit, to the information processing apparatus 103, an image signal of the subject that is generated by the solid state image sensor 11 by performing imaging, and peripheries of the plurality of signal cables 40 are covered by a shield 43 and the outer casing 44. elastic body includes, as illustrated in FIG. 3A to FIG. 3D, a first fixing portion 31 that is arranged. on the surface of the substrate 20 on which the electrodes 20a are formed, and a second fixing portion 32 that is arranged on the proximal end side relative to the substrate 20.

On the proximal end side of the first fixing portion 31, as illustrated in FIG. 2B and FIG. 3B, a plurality of grooves 33 for housing the plurality of signal cables 40 are formed. The grooves 33 are divided by wall portions 37, and are formed such that the electrodes 20a of the substrate 20 are located in. the grooves 33. The second fixing portion 32 includes, as illustrated in FIG. 2C, FIG. 3C, and FIG. 3D, a mounting portion 34 which has a U shape that conforms to an outer shape of the end portion of the composite cable 50 and on which the composite cable 50 is mounted.

A method of manufacturing the endoscope distal end structure 1 will be described below. First, the cable fixing portion 30 is formed, by molding, on the substrate 20 of the imaging unit 10, in particular, on the surface on which the plurality of electrodes 20a are formed, and on the proximal end side of the substrate 20.

After forming the cable fixing portion 30, the plurality of signal cables 40 that are exposed from the end portion of the composite cable 50 are arranged on the first fixing portion 31 of the cable fixing portion 30, and the end portion of the composite cable 50 is arranged on the mounting portion 34 of the second fixing portion 32.

After arranging the composite cable 50 on the cable fixing portion 30, core wires 41 of the signal cables 40 are connected to the respective electrodes 20a of the substrate 20 by conductive members.

After connecting the core wires 41, connection portions between the core wires 41 of the signal cables 40 and the electrodes 20a are filled with the sealing resin 36. It is sufficient to fill peripheries of the connection portions between the core wires 41 and the electrodes 20a with the sealing resin 36, but it may be possible to fill a periphery of the shield 43 that is exposed. from the outer casing 44 with the sealing resin 36.

After sealing the connection portions with the sealing resin 36, peripheries of the solid state image sensor 11 and the cable fixing portion 30 are covered by the heat shrinkable tube 22, the heat shrinkable tube 22 is shrunk, and the peripheries of the solid state image sensor 11 and the cable fixing portion 30 are coated by the heat shrinkable tube 22. When the heat shrinkable tube 22 is shrunk, the second fixing portion 32 that covers the periphery of the composite cable 50 is elastically deformed in a direction in which a space in the periphery of the composite cable 50 is reduced as illustrated in FIG. 3G. Meanwhile, in the present embodiment, the heat shrinkable tube that is shrunk by heat is used, but embodiments are not limited to this example as long as it is possible to ensure air tightness by shrinking.

In the endoscope distal end structure 1 according to the first embodiment, with use of the cable fixing portion 30, the composite cable 50 and the signal cables 40 are held by the cable fixing portion 30, so that it is possible to reduce a stress, such as blast, that is applied to the signal cables 40 and the composite cable 50. Further, it is possible to reduce a use amount of the sealing resin 36 that is used to seal the connection portions, and it is possible to prevent, by the cable fixing portion 30, the sealing resin 36 from being sucked up toward the composite cable 50 side, so that it is possible to easily control a length of a rigid portion of the endoscope 102. Furthermore, the grooves 33 are arranged in the first fixing portion 31 of the cable fixing portion 30, so that it is possible to easily determine positions of the signal cables 40.

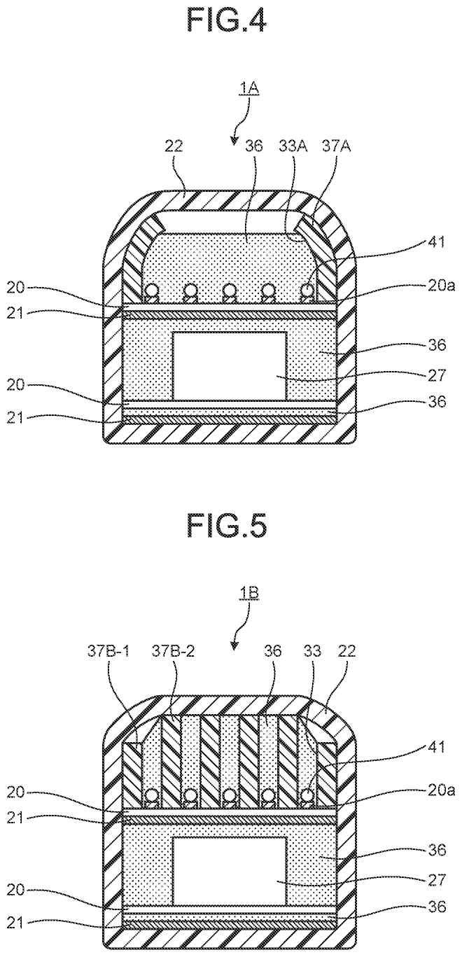

Meanwhile, in the first embodiment, the plurality of grooves 33 in which the signal cables 40 are housed are arranged in the first fixing portion 31 of the cable fitting portion 30, but embodiments are not limited to this example. FIG. 4 is a cross-sectional view of an endoscope distal end structure 1A according to a first modification of the first embodiment of the disclosure.

In the endoscope distal end structure 1A, a single large groove 33A is formed by a wall portion 37A, and all of the electrodes 20a are housed in the groove 33A. Even in the first modification of the first embodiment, it is possible to reduce the use amount of the sealing resin 36, and it is possible to prevent, by the cable fixing portion, the sealing resin 36 from being sucked up toward the composite cable 50 side, so that it is possible to easily control the length of the rigid portion of the endoscope 102.

Moreover, in the first embodiment, the wall portions 37 that form the grooves 33 of the cable fixing portion 30 have the same heights, but the heights of the wall portions 37 may be changed appropriately. FIG. 5 is a cross-sectional view of an endoscope distal end structure 1B according to a second modification of the first embodiment of the disclosure.

The endoscope distal end structure 1B is formed such that wall portions 37B-1 on both sides have lower heights than wall portions 37B-2 in a central portion. Even in the second modification of the first embodiment, it is possible to reduce the use amount of the sealing resin 36, and it is possible to prevent, by the cable fixing portion, the sealing resin 36 from being sucked up toward the composite cable 50 side, so that it is possible to easily control the length of the rigid portion of the endoscope 102.

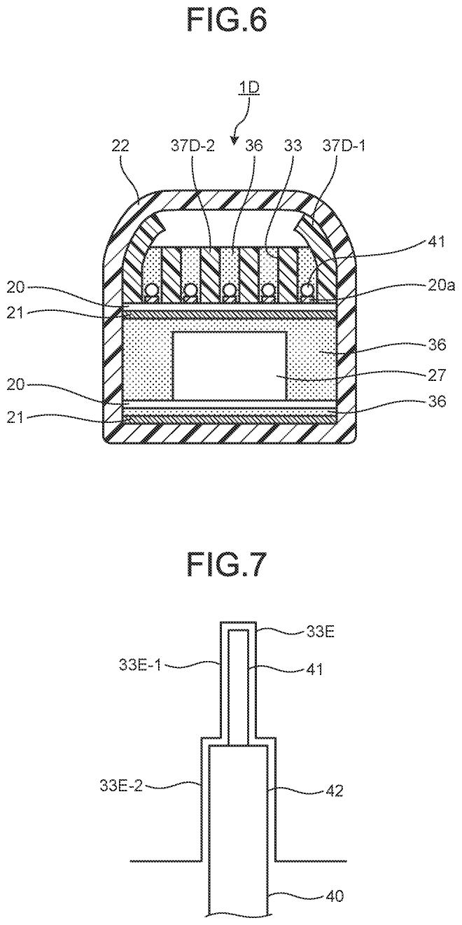

FIG. 6 is a cross-sectional view of an endoscope distal end structure 1D according to a third modification of the first embodiment of the disclosure. The endoscope distal end structure 11, is formed such that wall portions 37D-1 on both sides have higher heights than wall portions 37D-2 in a central portion. Even in the third modification of the first embodiment, it is possible to reduce the use amount of the sealing resin 36, and it is possible to prevent, by the cable fixing portion, the sealing resin 36 from being sucked up toward. the composite cable 50 side, so that it is possible to easily control the length of the rigid portion of the endoscope 102.

Furthermore, in the first embodiment, each of the grooves 33 has a rectangular shape that can house each of the signal cables 40, but embodiments are not limited to this example. FIG. 7 is an enlarged top view of a groove in a cable fixing portion of an endoscope distal end structure according to a fourth modification of the first embodiment of the disclosure.

In the endoscope distal end structure according to the fourth modification, a groove 33E is formed. of a first groove 33E-1 for housing each of the exposed core wires 41 of the signal cables 40, and a second groove 33E-2 for housing each of the signal cables 40 that are covered by electrical insulating bodies 42. The first groove 33E-1 has a rectangular shape that is slightly larger than an outer shape of each of the core wires 41, and the second groove 33E-2 has a rectangular shape that is slightly larger than an outer shape of each of the electrical insulating bodies 42. The groove 33E is formed of the first groove 33E-1 and the second groove 33E-2, so that it is possible to more easily determine the position of each of the signal cables 40 with respect to each of the electrodes 20a. In addition, it is possible to further reduce the use amount of the sealing resin, so that it is possible to further prevent the sealing resin 36 from being sucked up toward the composite cable 50 side.

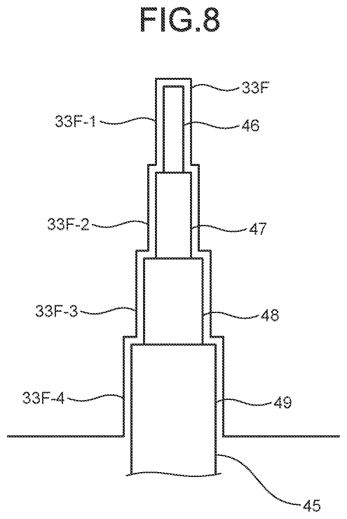

FIG. 8 is an enlarged top view of a groove in a cable fixing portion of an endoscope distal end structure according to a fifth modification of the first embodiment of the disclosure. In the endoscope distal end structure according to the fifth modification, the composite cable is formed by covering a plurality of coaxial cables 45 by an outer casing.

In the endoscope distal end structure according to the fifth modification, a groove 33F is formed of a first groove 33F-1 for housing each of exposed core wires 46 of the coaxial cables 45, a second. groove 33F-2 for housing each of exposed internal electrical insulators 47, a third groove 33F-3 for housing each of exposed shields 48, and a fourth groove- 33F-4 for housing each of the coaxial cables 45 that are covered by external electrical insulators 49. The first groove 33-F1 has a rectangular shape that is slightly larger than an outer shape of each of the core wires 46, the second groove 33F-2 has a rectangular shape that is slightly larger than an outer shape of each of the internal electrical insulators 47, the third. groove 33F-3 has a rectangular shape that is sightly larger than an outer shape of each of the shields 48, and the fourth groove 33F-4 has a rectangular shape that is slightly larger than an outer shape of each of the external electrical insulators 49. The groove 33F is formed of the first groove 33F-1, the second groove 33F-2, the third groove 33F-3, and the fourth groove 33F-4, so that it is possible to more easily determine a position of each of the coaxial cables 45 with respect to each of the electrodes. In addition, it is possible to further reduce the use amount of the sealing resin, so that it is possible to further prevent the sealing resin 36 from being sucked up toward the composite cable 50 side.

### Second Embodiment

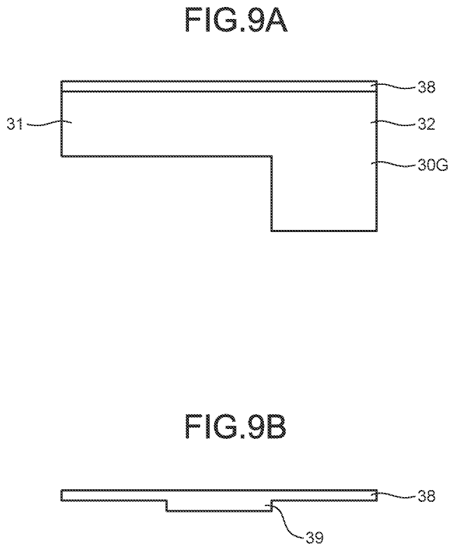

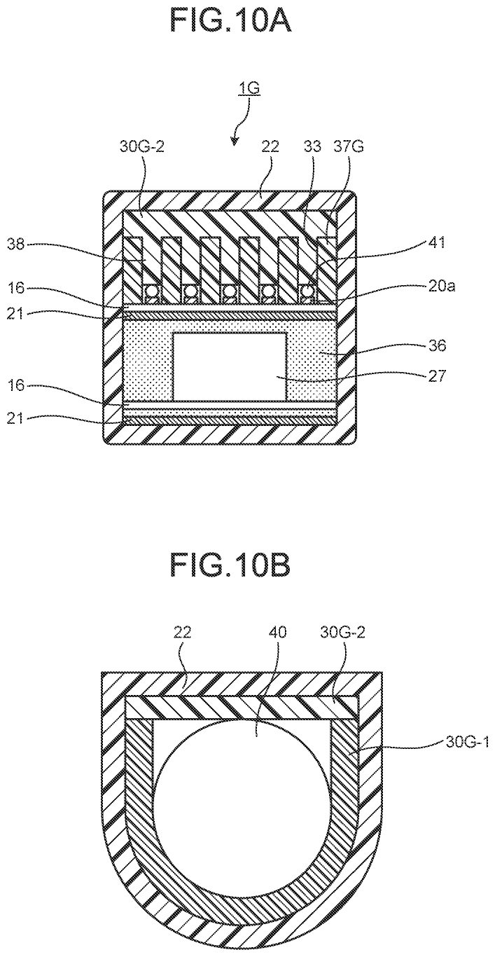

An endoscope distal end structure according to a second embodiment includes an upper cable fixing portion that is formed of an elastic body and that is arranged on an upper surface of the cable fixing portion. FIG. 9A is a side view of a cable fixing portion 30G and an upper cable fixing portion 38 that are used in an endoscope distal end structure 1G according to the second embodiment of the disclosure. FIG. 9B is a side view of the upper cable fixing portion 38. FIG. 10A is a cross-sectional view of the endoscope distal end structure 1G according to the second embodiment of the disclosure (cross section at the first fixing portion 31). FIG. 10B is a cross-sectional view of the endoscope distal end structure 1G according to the second embodiment of the disclosure (cross section at the second fixing portion 32).

The upper cable fixing portion 38 is arranged on the upper surface of the cable fixing portion 30G, and holds, with the cable fixing portion 30G, the plurality of signal cables 40 that are exposed from the end portion of the composite cable 50 and the end portion of the composite cable 50. The cable fixing portion 30G includes, similarly to the cable fixing portion 30 of the first embodiment, the first fixing portion 31 with the grooves 33, and the second fixing portion 32.

The upper cable fixing portion 38 includes, as illustrated in FIG. 9B and FIG. 10A, protrusions 39 that are fitted to the grooves 33, on a surface that comes into contact with the cable fixing portion 30G. The protrusions are fitted in the grooves 33, and come into contact with the connection portions between the core wires 41 and the electrodes 20a In the endoscope distal end structure 1G, the protrusions of the upper cable fixing portion 38 that is an elastic body come into contact with and protect the connection portions between the core wires 41 and the electrodes 20a, and therefore, the connection portions between the core wires 41 and the electrodes 20a are not filled with a sealing resin. Therefore, sucking up of the sealing resin toward the composite cable 50 side does not occur, and it is possible to reduce the length of the rigid portion of the endoscope.

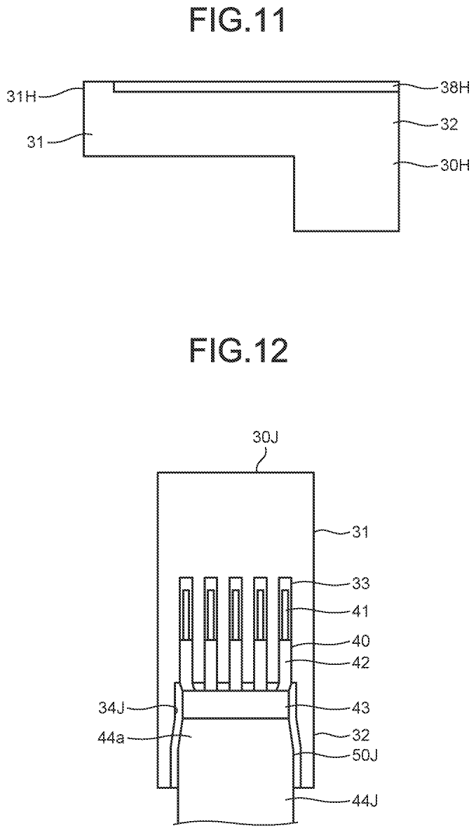

In the second embodiment, lengths of the upper cable fixing portion 38 and the cable fixing portion 30G in the optical axis direction are set to the same, but embodiments are not limited to this example. FIG. 11 is a side view of a cable fixing portion 30H and an upper cable fixing portion 38H that are used in an endoscope distal end, structure according to a modification of the second embodiment of the disclosure.

The cable fixing portion 30H includes, at a side of the image sensor, a butt portion 31H that buts against the upper cable fixing portion 38H for positioning. In the cable fixing portion 30H, when the upper cable fixing portion 38H is to be placed on the upper surface of the cable fixing portion 30H, it is possible to cause the upper cable fixing portion 38H to butt against the the butt portion 31H for positioning, so that it is possible to easily determine the position. Furthermore, with arrangement of the butt portion 31H, it is possible to reduce a bad influence on the image sensor due to oscillation that is caused when the upper cable fixing portion 38H is placed on the cable fixing portion 30H.

### Third Embodiment

An endoscope distal end structure according to a third embodiment is configured such that a mounting portion of a second fixing portion for mounting a composite cable has a tapered shape FIG. 12 is a top view of the endoscope distal end structure according to the third embodiment of the disclosure.

In the endoscope distal end. structure according to the third embodiment, an outer casing 44J at a side of an end portion of a composite cable 50J has a tapered shape in which an end portion has a reduced diameter, and a mounting portion 34J in a second fixing portion of a cable fixing portion 30J is also tapered so as to conform to the tapered shape of the outer casing 44J. By forming the outer casing 44J and the mounting portion 34J in the tapered shapes, it is possible to reduce a gap between the composite cable 50 and the cable fixing portion 30J, so that it is possible to prevent sucking up of the sealing resin.

According to one aspect of the disclosure, it is possible to obtain an endoscope distal end structure with a reduced length of a rigid portion.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope distal end structure comprising:
an image sensor configured to capture an image of an inside of a subject and generate an image signal;
a composite cable that is formed by bundling a plurality of signal cables by an electrical insulating outer casing;
a substrate that includes a plurality of electrodes for connecting respective core wires of the plurality of signal cables, the substrate being configured to electrically connect the image sensor and the plurality of signal cables;
a cable fixing portion that is formed of an elastic body, the cable fixing portion being configured to hold the plurality of signal cables that are exposed from an end portion of the composite cable and the end portion of the composite cable;
a sealing resin configured to protect connection portions between the core wires of the plurality of signal cables and the plurality of electrodes; and
a shrinkable tube configured to cover peripheries of the image sensor and the cable fixing portion, wherein
the cable fixing portion includes
a first fixing portion that is arranged on a surface of the substrate, the electrodes are formed on the surface of the substrate, the first fixing portion being configured to cover at least a part of peripheries of the plurality of signal cables, and
the first fixing portion includes grooves that are divided by wall portions, the grooves being configured to house the respective signal cables.

2. The endoscope distal end structure according to claim 1, wherein the cable fixing portion includes a second fixing portion which is arranged on a proximal end side relative to the substrate, on which the end portion of the composite cable is mounted, and which has a U shape that conforms to an outer shape of the end portion of the composite cable.

3. The endoscope distal end structure according to claim 1, further comprising:
an upper cable fixing portion that is formed of an elastic body, and that is arranged on an upper surface of the cable fixing portion, the upper cable fixing portion being configured to hold, with the cable fixing portion, the plurality of signal cables that are exposed from the end portion of the composite cable and the end portion of the composite cable, wherein
protrusions that fitted to the grooves are arranged on a surface of the upper cable fixing portion, the surface of the upper cable fixing portion being in contact with the cable fixing portion, and
the connection portions between the core wires of the plurality of signal cables and the plurality of electrodes are not filled with a sealing resin.

4. The endoscope distal end structure according to claim 3, wherein the cable fixing portion includes, at a side of the image sensor, a butt portion configured to butt against the upper cable fixing portion for positioning.

5. The endoscope distal end structure according to claim 1, wherein
the outer casing of the end portion of the composite cable has a tapered shape in which an end portion has a reduced diameter,
the cable fixing portion includes a second fixing portion which. is arranged on a proximal end side relative to the substrate and on which the end portion of the composite cable is mounted, and
a mounting portion of the second fixing portion has a tapered shape that conforms to an outer shape of the end portion of the composite cable, the mounting portion being configured to mount the composite cable thereon.

6. An endoscope comprising:
an insertion portion configured to be inserted into a subject;
an image sensor that is arranged on the insertion portion, the image sensor being configured to capture an image of an inside of the subject and generate an image signal;
a composite cable that is formed by bundling a plurality of signal cables by an electrical insulating outer casing;
a substrate that includes a plurality of electrodes for connecting respective core wires of the plurality of signal cables, the substrate being configured to electrically connect the image sensor and the plurality of signal cables;
a cable fixing portion that is formed of an elastic body, the cable fixing portion being configured to hold the plurality of signal cables that are exposed from as end portion of the composite cable and the end portion of the composite cable;
a sealing resin configured to protect connection portions between the core wires of the plurality of signal cables and the plurality of electrodes; and
a shrinkable tube configured to cover peripheries of the image sensor and the cable fixing portion, wherein
the cable fixing portion includes
a first fixing portion that is arranged on a surface of the substrate, the electrodes being formed on the surface of the substrate, the first fixing portion being configured to cover at least a part of peripheries of the plurality of signal cables, and
the first fixing portion includes grooves that are divided by wall portions, the grooves being configured to house the respective signal cables.

7. The endoscope according to claim 6, wherein the cable fixing portion includes a second fixing portion which is arranged on a proximal end side relative to the substrate, on which the end portion of the composite cable is mounted, and which has a U shape that conforms to an outer shape of the end portion of the composite cable.

8. The endoscope according to claim 6, further comprising:
an upper cable fixing portion that is formed of an elastic body, and that is arranged on an upper surface of the cable fixing portion, the upper cable fixing portion being configured to hold, with the cable fixing portion, the plurality of signal cables that are exposed from the end portion of the composite cable and the end portion of the composite cable, wherein
protrusions that are fitted to the grooves are arranged on a surface of the upper cable fixing portion, the surface of the upper cable fixing portion being in contact with the cable fixing portion, and
the connection portions between the core wires of the plurality of signal cables and the plurality of electrodes are not filled with a sealing resin.

9. The endoscope according to claim 8, wherein the cable fixing portion includes, at a side of the image sensor, a butt portion configured to butt against the upper cable fixing portion for positioning.

10. The endoscope according to claim 9, wherein
the outer casing of the end portion of the composite cable has a tapered shape in which an end portion has a reduced. diameter,
the cable fixing portion includes a second fixing portion which is arranged on a proximal end side relative to the substrate and on which the end portion of the composite cable is mounted, and
a mounting portion of the second fixing portion has a tapered shape that conforms to an outer shape of the end portion of the composite cable, the mounting portion being configured to mount the composite cable thereon.
